(19) United States
(12) Patent Application Publication
(10) Pub. No.: US 2020/0375572 A1
(43) Pub. Date: Dec. 3, 2020

(54) CLINICAL DATA ACQUISITION SYSTEM WITH MOBILE CLINICAL VIEWING DEVICE

(71) Applicant: EchoNous, Inc., Redmond, WA (US)

(72) Inventors: Donald James Rainville, Sewickley, PA (US); Bradley Scott Melmon, Seattle, WA (US); Greg Nieminen, Bothell, WA (US); Nikolaos Pagoulatos, Kirkland, WA (US); Nidhi Jaiswal, Redmond, WA (US); Qianying Miao, Redmond, WA (US)

(21) Appl. No.: 16/888,372

(22) Filed: May 29, 2020

Related U.S. Application Data
(60) Provisional application No. 62/854,931, filed on May 30, 2019.

(51) Int. Cl. A61B 8/00; A61B 8/08; A61B 8/14; G01N 29/06; G01N 29/22

(52) U.S. Cl. CPC A61B 8/4427; A61B 8/4472; G01N 29/226; A61B 8/14; G01N 29/0645; A61B 8/5207

(57) ABSTRACT

A clinical data acquisition system includes a probe and a mobile clinical viewing device communicatively coupleable to the probe. The probe includes at least one sensor that acquires physiological data of a patient. The mobile clinical viewing device includes a frame, a display, and a handle. The display is secured to a first side of the frame. The handle is secured to a second side of the frame. The handle includes a plurality of user input elements, and a user may control one or more operations of the mobile clinical viewing device by providing input via the user input elements on the handle. This allows a user to comfortably hold the mobile clinical viewing device and to control operations of the device in one hand, while the other hand is free to perform other operations such as holding the probe during examination of the patient.

### BACKGROUND

### Technical Field

The present application pertains to clinical data acquisition systems, such as ultrasound systems, and more particularly to ultrasound systems with a mobile clinical viewing device for displaying ultrasound images during ultrasound imaging.

### Description of the Related Art

Ultrasound imaging is a useful imaging modality in a number of environments. For example, in the field of healthcare, internal structures of a patient's body may be imaged before, during or after a therapeutic intervention. A healthcare professional may hold a portable ultrasound probe in proximity to the patient and move the transducer as appropriate to visualize one or more target structures in a region of interest in the patient. The healthcare professional coordinates the movement of the probe so as to obtain a desired representation on a screen, such as a two-dimensional cross-section of a three-dimensional volume.

Ultrasound may also be used to measure functional aspects of a patient, such as organ movement and blood flow in the patient. Doppler measurements, for example, are effective in measuring the direction and speed of movement of a structure, such as a heart valve or blood cells flowing in a vessel, relative to the transducer. Doppler echocardiography is widely used for evaluating the cardiocirculatory system of patients with known or suspected cardiovascular disease.

For many years, ultrasound imaging was effectively confined to large equipment operating in a hospital environment. Recent technological advances, however, have produced smaller ultrasound systems that increasingly are deployed in frontline point-of-care environments, e.g., doctor's offices.

### BRIEF SUMMARY

The present application, in part, addresses a desire for smaller clinical data acquisition systems (e.g., ultrasound systems) having greater portability, lower cost, and ease of use for different modes of data acquisition (e.g., different modes of ultrasound imaging), while at the same time providing high quality measurements and user-friendly features for controlling an operation of the clinical data acquisition systems, such as by manipulating various ultrasound imaging or other data acquisition parameters.

In at least one embodiment, a mobile clinical viewing device includes a frame, a display, and a handle. The display is secured to a first side of the frame. The handle is secured to a second side of the frame. The handle includes a plurality of user input elements which are used to control one or more operations of the mobile clinical viewing device in response to received input from a user.

In at least one embodiment, a clinical data acquisition system is provided that includes a probe and a mobile clinical viewing device communicatively coupleable to the probe. The probe includes at least one sensor configured to acquire physiological data of a patient. The mobile clinical viewing device includes a frame, a display, and a handle. The display is secured to a first side of the frame. The handle is secured to a second side of the frame which may be opposite the first side of the frame. The handle includes a plurality of user input elements, such as buttons, sliders, or any element capable of receiving user input. In use, a user may control one or more operations of the mobile clinical viewing device by providing input via the user input elements on the handle, e.g., using the same hand that is holding the handle. This allows a user to comfortably hold the mobile clinical viewing device and to control operations of the device in one hand, while the other hand is free to perform other operations that do not involve manipulation of the clinical viewing device, such as holding the probe during examination of the patient.

In at least one embodiment, an ultrasound system is provided that includes an ultrasound probe configured to acquire ultrasound data of a patient, and a mobile clinical viewing device communicatively coupleable to the ultrasound probe. The mobile clinical viewing device includes a display configured to display ultrasound images based on the acquired ultrasound data of the patient, and a handle coupled to a back side of the display. The handle includes a plurality of user input elements configured to control one or more operations of the mobile clinical viewing device in response to input received from a user.

### BRIEF DESCRIPTION OF THE SEVERAL VIEWS OF THE DRAWINGS

FIG. 1A is a side view illustrating an ultrasound system that includes a mobile clinical viewing device and a clinical data acquisition probe, in accordance with one or more embodiments of the present disclosure.

FIG. 1B is a perspective bottom view illustrating the ultrasound system of FIGS. 1A and 1B, in accordance with one or more embodiments of the present disclosure.

FIG. 2A is a top view illustrating the mobile clinical viewing device of the ultrasound system shown in FIGS. 1A and 1B, in accordance with one or more embodiments.

FIG. 2B is a bottom view illustrating the mobile clinical viewing device of the ultrasound system shown in FIGS. 1A and 1B, in accordance with one or more embodiments.

FIG. 2C is a right side view illustrating the mobile clinical viewing device of the ultrasound system shown in FIGS. 1A and 1B, in accordance with one or more embodiments.

FIG. 2D is a left side view illustrating the mobile clinical viewing device of the ultrasound system shown in FIGS. 1A and 1B, in accordance with one or more embodiments.

FIG. 2E is a back side view illustrating the mobile clinical viewing device of the ultrasound system shown in FIGS. 1A and 1B, in accordance with one or more embodiments.

FIG. 2F is a front side view illustrating the mobile clinical viewing device of the ultrasound system shown in FIGS. 1A and 1B, in accordance with one or more embodiments.

FIG. 2G is a perspective bottom view illustrating the mobile clinical viewing device of the ultrasound system shown in FIGS. 1A and 1B, in accordance with one or more embodiments.

FIG. 2H is a left side view illustrating the mobile clinical viewing device of the ultrasound system shown in FIGS. 1A and 1B in a resting state on a flat surface, in accordance with one or more embodiments.

FIG. 3A is a perspective top view illustrating the clinical data acquisition probe of the ultrasound system shown in FIGS. 1A and 1B, in accordance with one or more embodiments.

FIG. 3B is a side view illustrating the clinical data acquisition probe of the ultrasound system shown in FIGS. 1A and 1B, in accordance with one or more embodiments.

### DETAILED DESCRIPTION

Off-the-shelf mobile devices, such as smart phones, tablet computing devices or the like, are typically not optimal for use in clinical data acquisition systems, such as ultrasound imaging systems. One reason for this is that in many clinical scenarios, the user typically holds the mobile device with one hand and while the other hand is used to scan the patient, for example, with a probe such as an ultrasound probe. The user generally uses both hands to manipulate the mobile device, such as to provide user input through a touch screen or selectable elements on the display of the mobile device. As such, the user cannot typically manipulate the mobile device or provide input via the mobile device while simultaneously holding the probe to scan a patient.

In various embodiments, the present disclosure provides a highly mobile clinical data acquisition system (such as an ultrasound system) which includes a probe and a mobile computing device or system for control, viewing and additional computation, which is also highly mobile and handheld in the form factor of a phone or a tablet. This allows performing medical examinations, such as ultrasound examinations, on the go at any and all locations.

Current off-the-shelf mobile device (phone or tablet) solutions do not meet the needs in the clinical scenarios mentioned above, where the user is holding the mobile device with one hand and the probe with the other hand. If the mobile device used is above a certain size and is either a large phone or a tablet (which may be desirable due to the larger screen compared to a small phone), there is no optimal way for the user to comfortably and securely hold the mobile device with one hand while using the other hand to scan with the probe and at the same time having the ability to control key imaging functions by interacting with the tablet. Even by adding a case to the mobile device, which may address issues relating to comfort and secure holding with one hand, control of the probe and the mobile device is still a problem because the user cannot access the mobile device touchscreen or other control inputs of the mobile device since both hands are occupied. Likewise, off-the-shelf mobile devices cannot be securely positioned on a flat surface at an upright angle without a case or additional accessories. Described herein is a clinical data acquisition system (which in some embodiments may be an ultrasound system) that includes a mobile clinical viewing device (which may be referred to herein as a tablet), which may be any mobile computing device, display device, or the like which is operatively coupled to a portable probe, such as an ultrasound probe. The tablet includes a handle which may be used by a user to hold the tablet, for example, during use of the ultrasound system to perform ultrasound imaging. The handle includes user input elements through which the user may provide input to control operations of the ultrasound system.

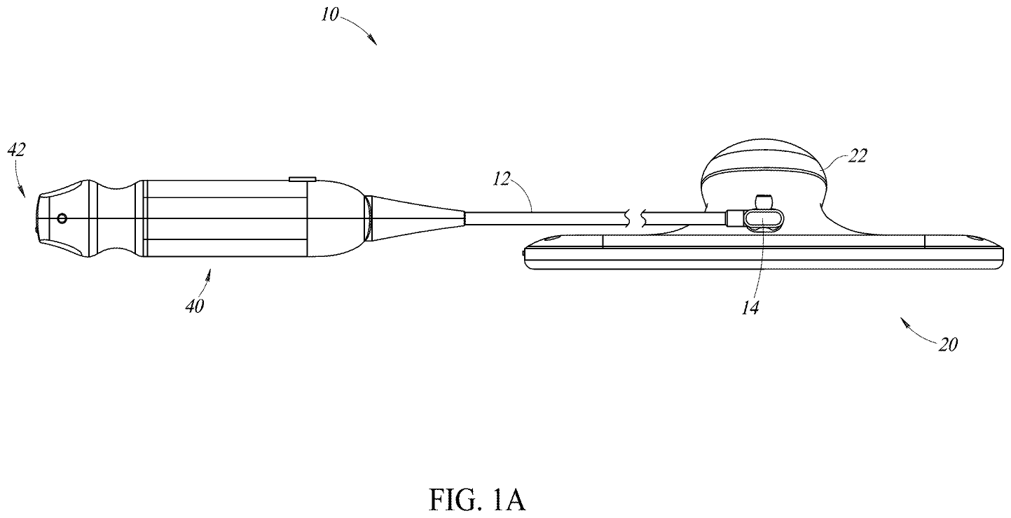

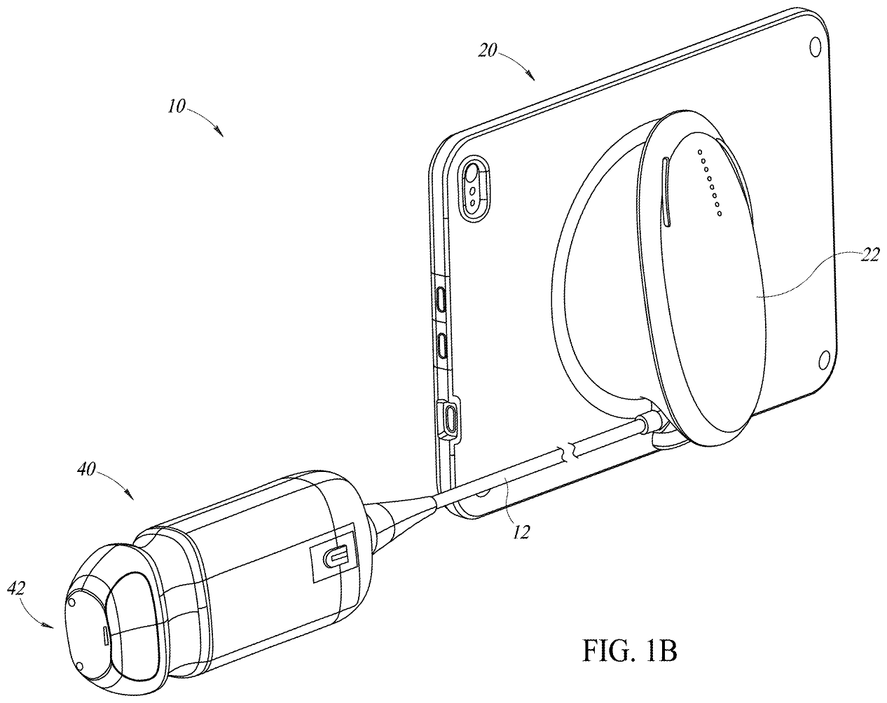

FIG. 1A is a side view illustrating a clinical data acquisition system 10 in accordance with one or more embodiments of the present disclosure. FIG. 1B is a perspective rear view illustrating the clinical data acquisition system 10 in accordance with one or more embodiments of the present disclosure.

Referring to FIGS. 1A and 1B, the clinical data acquisition system 10 includes a mobile clinical viewing device 20 (which may be referred to herein as tablet 20) and a clinical data acquisition probe 40 (which may be an ultrasound probe and may be referred to herein as ultrasound probe 40). The mobile clinical viewing device 20 may be or include any mobile, handheld computing device having a display, including, for example, a tablet computer, a smart phone, or the like. The mobile clinical viewing device 20 includes a handle 22 which provides an ergonomic gripping structure for securely holding, controlling, and manipulating the mobile clinical viewing device 20 during use by a user.

The ultrasound probe 40 is electrically coupled to the tablet 20 by a cable 12. The cable 12 includes a connector 14 that detachably connects the probe 40 to the tablet 20. The cable 12 facilitates bi-directional communication between the tablet 20 and the probe 40.

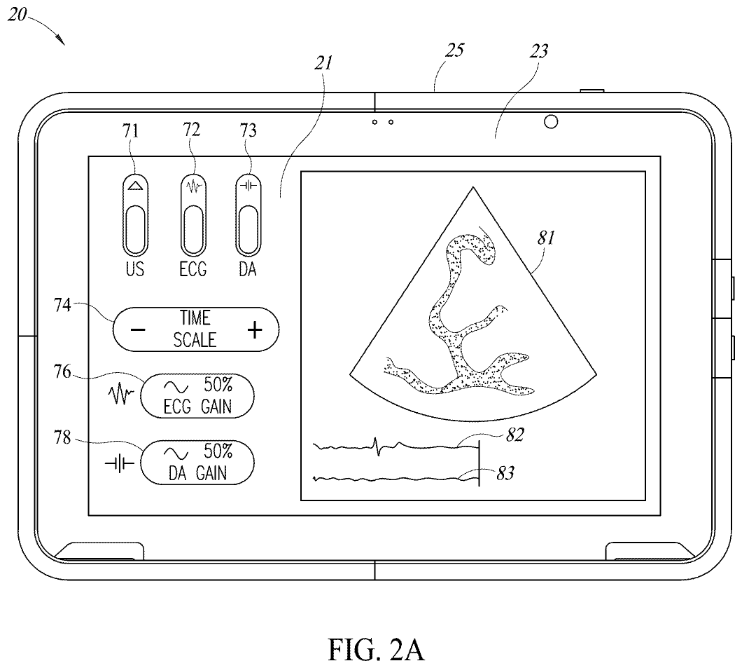

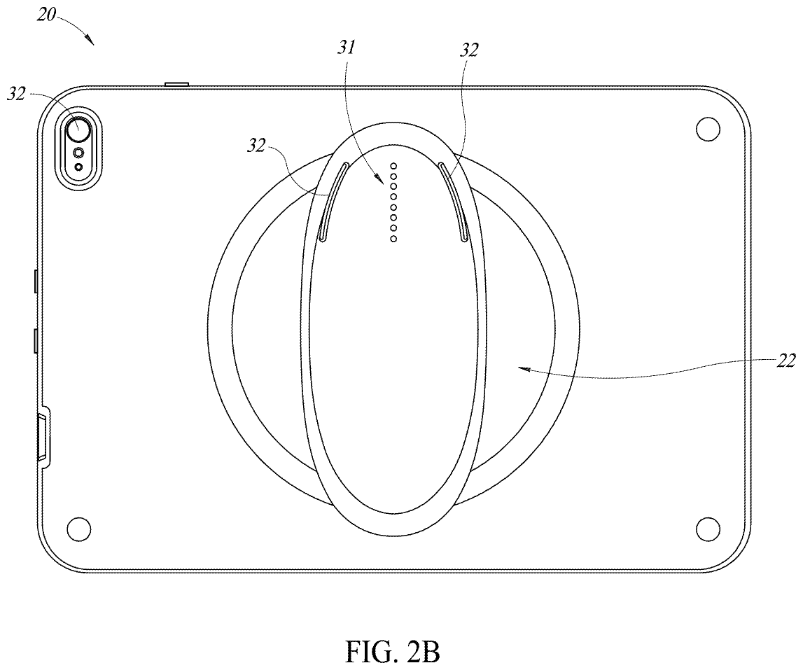

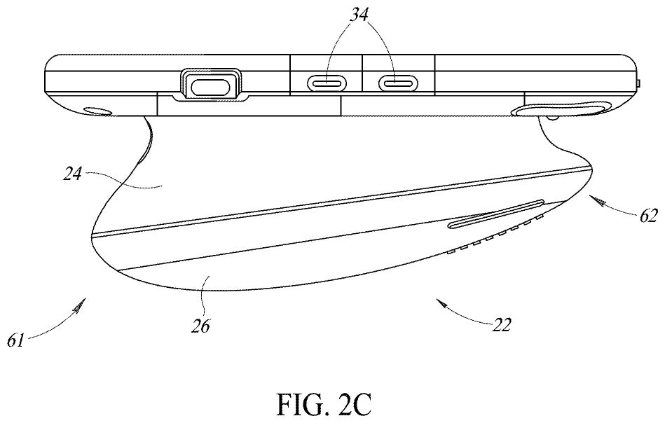

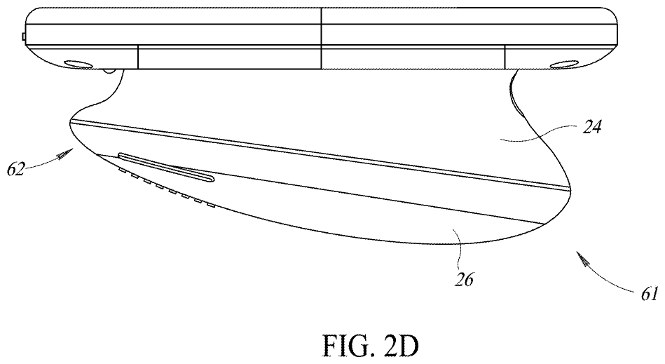

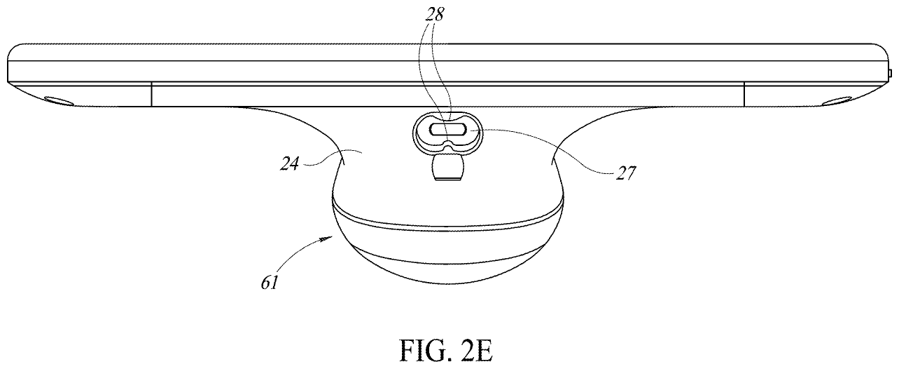

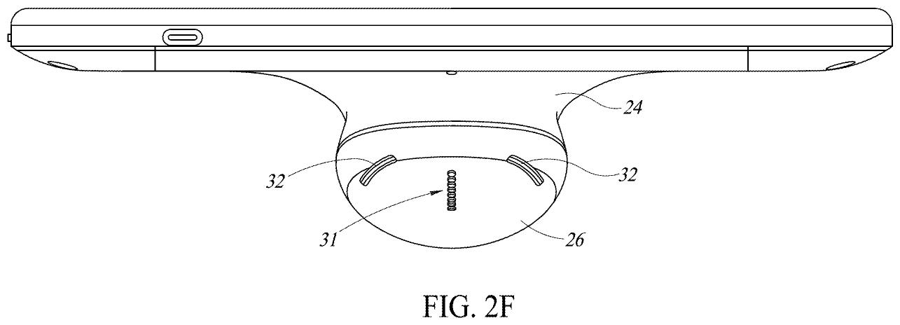

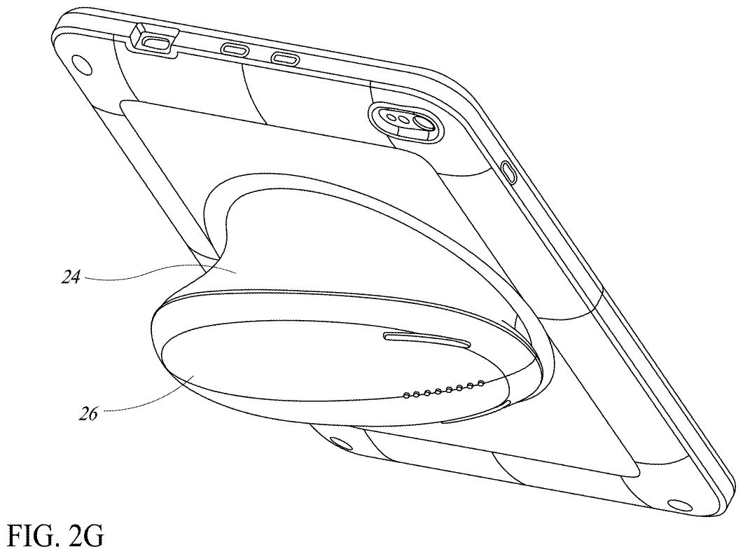

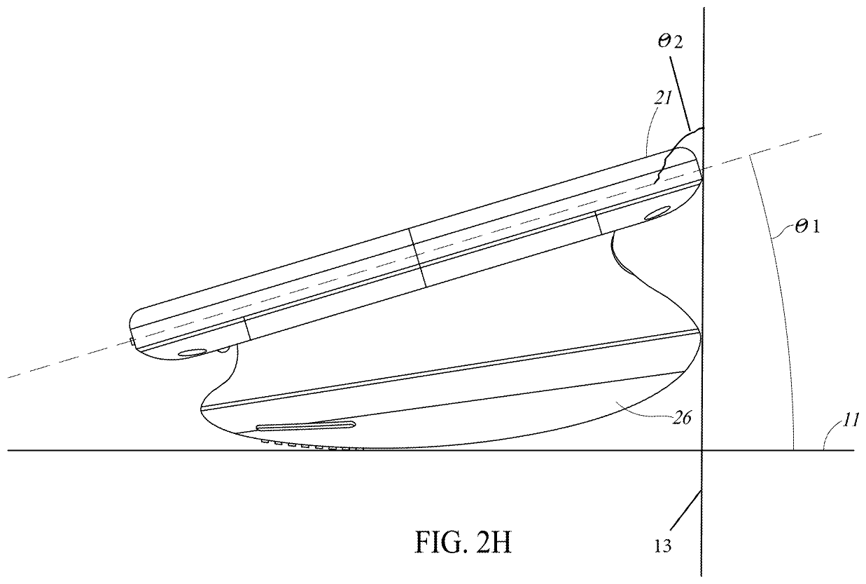

FIGS. 2A through 2H are various views illustrating the tablet 20 of the clinical data acquisition system 10, in accordance with one or more embodiments.

Referring to FIGS. 2A through 2H, the tablet 20 includes a display 21, a frame 23, and an overmolded outer layer 25 (which may be referred to herein as an overmold). The display 21 may be used, for example, to display clinical imagery, such as ultrasound images, during or after imaging of a patient. The frame 23 generally forms a housing within which electronic components of the tablet 20 may be contained, and in some embodiments, the frame 23 surrounds edge portions of the display 21. In some embodiments, the frame 23 may include magnesium, or the frame 23 may be a magnesium frame. Magnesium provides good stiffness for the frame 23, and also has low weight.

The overmold 25 may be a silicon rubber overmold in some embodiments. The overmold 25 is formed directly on the tablet 20. For example, the overmold 25 may be formed directly on exposed surfaces of the frame 23. The overmold 25 may be formed by an overmolding process in which the overmold 25 is formed directly on the frame 23.

The handle 22 is located on a side of the tablet 20, e.g., as shown in FIG. 2B on a side that is opposite the display 21. For example, the display 21 may be located on a front or upper side of the tablet 20 (as shown, for example, in the top view of FIG. 2A), while the handle 22 may be located on a back or lower side of the tablet 20 (as shown, for example, in the bottom view of FIG. 2B). In this way, the user may grip the handle 22 on a back side of the tablet 20, and the display 21 on the front or top side of the tablet 20 may face upward toward the user so that the user may have an unobstructed view of the display 21. In some embodiments, e.g., as shown in FIG. 2C, the handle 22 includes a lower portion 24 (e.g., closer in proximity to the back side of the tablet 20) and an upper portion 26 (e.g., farther from the back side of the tablet 20). The lower portion 24 extends outwardly from the back side of the frame 23. For example, the lower portion 24 may extend downward from a back surface of the frame 23. In some embodiments, the lower portion 24 may be directly connected to the frame 23, for example, with the lower portion 24 of the handle 22 extending directly outwardly from the frame 23.

The lower portion 24 of the handle 22 may have a curved shape, which may provide for comfortable and ergonomic gripping of the handle 22 by the hand of a user. In some embodiments, the lower portion 24 of the handle 22 is formed of a material that is different from that of the frame 23. In some embodiments, the lower portion 24 of the handle 22 is formed of or includes aluminum. Aluminum generally provides good ruggedness and good heat dissipation properties for the lower portion 24 of the handle 22.

The upper portion 26 of the handle 22 may be formed of a different material than the lower portion 24. For example, in some embodiments, the upper portion 26 of the handle 22 is overmolded silicone, which may facilitate comfortable holding and secure gripping by the user. In some embodiments, the upper portion 26 may cover a part of the lower portion 24 of the handle 22. For example, the lower portion 24 may substantially define a shape of the handle 22, while the upper portion 26 may cover only part of the lower portion 24 of the handle 22, such as covering a part of the handle 22 which, in use, is contacted by a palm of a user's hand.

In some embodiments, the upper portion 26 of the handle 22 includes an inner shell which may be attached to an end of the lower portion 24. The inner shell may be formed of a rigid material, such as a rigid plastic. The upper portion 26 of the handle 22 may further include an outer shell which covers the inner shell, and which is formed of a softer material, such as silicone or the like. The use of silicone in the outer shell provides improved grip, chemical resistance, and better feel and control when held by a user.

In some embodiments, the silicone outer shell of the upper portion 26 of the handle 22 helps to stabilize the tablet 20 in a particular orientation when the tablet 20 is placed on a flat surface, such as a table. This is illustrated, for example, in FIG. 2H. As shown in FIG. 2H, when the tablet 20 is placed face up on a flat surface 11 (e.g., with the upper portion 26 of the handle 22 in contact with the surface 11), the tablet 20 may be balanced in a position at which the display 21 has a non-zero inclination angle 81 with respect to the surface 11. In various embodiments, the display 21 may have any inclination angle 81 that is between 0° and 90°. In some embodiments, the display 21 has an inclination angle 81 between 20° and 70°. In some embodiments, display 21 has an inclination angle 81 that is between 30° and 60°. The inclination angle 81 of the display 21 provides a convenient viewing angle for the user when the tablet 20 is placed on a flat surface. In some embodiments, the handle 22 includes an internal articulating mechanism that allows the user to extend or retract an end of the handle 22 (e.g., a back end 61 or front end 62) relative to the back side of the display 21, and thereby manually or automatically (e.g., by electrical control of the internal articulating mechanism) adjust the inclination angle 81 of the display 21.

Additionally, as shown in FIG. 2H, the tablet 20 may be placed on a flat surface 13 and may have a non-zero inclination angle 82 with respect to the surface 13. For example, the flat surface 13 may be a horizontal surface, and the tablet 20 may be placed on the surface 13 with the back end 61 of the handle 22 and a rear portion of the display 21 (or rear portions of the frame 23 or overmold 25 covering the display 21) resting on the surface 13. In various embodiments, the display 21 may have any inclination angle 82 that is between 90° and 180° (e.g., as measured between the flat surface 13 and the viewing surface of the display 21). In some embodiments, the display 21 has an inclination angle 82 between 90° and 135°. In some embodiments, display 21 has an inclination angle 82 that is between 100° and 125°. The inclination angle 82 of the display 21 provides a convenient viewing angle for the user when the tablet 20 is placed on a flat surface 13.

In some embodiments, the display 21 may be rotatable with respect to the handle 22. For example, in some embodiments, the handle 22 may be rotatable about an axis that extends perpendicular to a display surface of the display 21. With reference to FIG. 2G, the handle 22 may rotate (e.g., clockwise or counterclockwise) about a rotational axis that extends through a center of the display 21, perpendicular to the surface of the display 21. Accordingly, in some embodiments, the user may rotate the display 21 with respect to the handle 22 in order to provide any desired viewing rotation of the display. In some embodiments, the user may rotate the display 21 in order to view the display in a portrait orientation or in a landscape orientation, as may be desired. In some embodiments, the display 21 may be rotatably coupled to the handle 22 and configured to freely rotate about the axis of rotation with respect to the handle 22. In some embodiments, the display 21 is rotatable with respect to the handle 22 within a selected or defined range. For example, in some embodiments, the display 21 may be rotated within a range between 0° and 90° in either the clockwise or counterclockwise directions.

The handle 22 provides the ability to securely and comfortably hold the tablet 20 with one hand while the other hand is free to perform other operations that do not involve manipulation of the tablet 20, such as to hold the probe 40 that is used to acquire physiological data, such as ultrasound, ECG, or auscultation data of the patient. The shape of the handle 22 is conducive to a secure and comfortable grip for a wide range of hand sizes and for both and left hands. In some embodiments, the handle 22 has a generally symmetrical shape about a long axis, as shown in FIG. 2B, which illustrates the handle 22 having a generally elliptical shape.

In some embodiments, the handle 22 slopes upwardly (e.g., inwardly toward the display 21) from a back end 61 of the handle toward a front end 62 of the handle. For example, as shown in FIGS. 2C and 2D, an outer surface of the upper portion 26 of the handle may be spaced farther away from the display 21 at the back end 61 than at the front end 62. In use, the user may position the open palm of a hand on or near the back end 61 of the handle 22, and the fingers may extend forward toward the front end 62.

The handle 22 further includes a plurality of user input elements which may be utilized by a user to manipulate parameters associated with clinical data acquisition (e.g., parameters for ultrasound imaging or the like) or associated with display of acquired clinical data (e.g., ECG data, auscultation data, or ultrasound images) or other features of the display 21. In some embodiments, the user input elements include a slider or scrolling feature 31 and one or more buttons 32. The slider 31 may be any input element which is capable of receiving a directional input (e.g., by a user sliding a finger upward or downward along the slider 31). The slider 31 may be, for example, a scrolling wheel or the like. In some embodiments, the slider 31 includes a plurality of separate sensors which are spaced apart from one another and which are aligned with one another along an axis of the slider 31. The separate sensors of the slider 31 may be any suitable sensor for receiving an input from a user, including, for example, capacitive sensors or the like that can sense touch.

In some embodiments, the slider 31 is disposed along a central axis (e.g., the long axis) of the handle 22, and two or more buttons 32 are spaced laterally apart from the slider 31. The buttons 32 may be any buttons, knobs, switches, or the like, capable of receiving input from a user of the tablet 20. In some embodiments, the buttons 32 may be capacitive sensors which receive input from the user, e.g., via the fingertips.

In some embodiments, a touch pad may be included as a user input element on the handle 22. For example, in some embodiments, a touch pad may be included as a user input element on the handle 22, for example, in place of the slider 31. The buttons 32 may be positioned on either side of the touch pad, and the touch pad may be capable of receiving user input from one or more fingers of the user.

Using the plurality of user input elements, a user can control or manipulate any parameters of the clinical data acquisition system 10, including data acquisition parameters (e.g., imaging-related parameters) as well as display-related parameters. For example, in some embodiments, the user may control via the user input elements one or more ultrasound imaging-related parameters such as depth, gain, freeze, saving an image or clip, controlling a region of interest, or any other ultrasound imaging-related parameters. In some embodiments, the user input elements may be utilized to control or adjust a gain of acquired clinical data, such as a gain of ECG data or digital auscultation data that is acquired by the probe 40.

In some embodiments, display-related parameters of the clinical data acquisition system 10 may be controlled via the user input elements, such as zooming in or out on an ultrasound image displayed on the display 21, scrolling through features or displayed images, making a selection of selectable elements displayed on the display 21, or the like. Advantageously, by arranging the user input elements on the handle 22, a user can control or manipulate parameters of the clinical data acquisition system 10 using the same hand that is holding the display 21, thus freeing the users other hand to perform other operations including control or manipulation of a probe 40 that acquires clinical data.

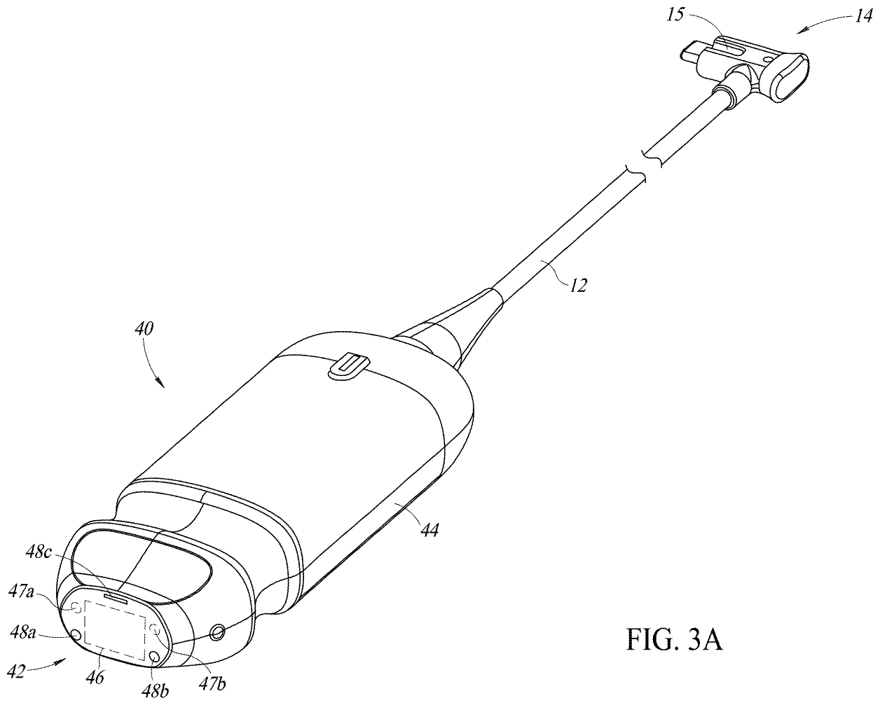

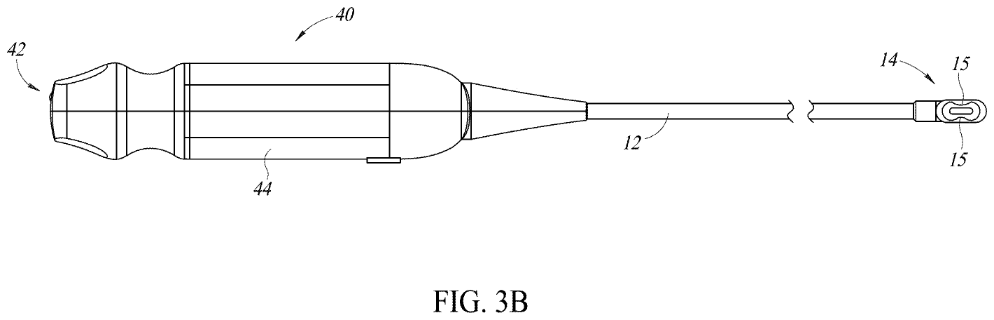

As shown in FIG. 2A, in some embodiments, the display 21 is configured to display a variety of selectable icons or features. For example, selectable icons 71, 72, 73 may be displayed for controlling display of ultrasound data 81, ECG data 82, and auscultation data 83, respectively. Through selection of any of the selectable icons 71, 72, 73 (e.g., by user input provided via the slider 31 or buttons 32), the ultrasound data 81, ECG data 82, and auscultation data 83 may be selectively displayed or omitted from display. In some embodiments, the selectable icons 71, 72, 73 may be utilized to control acquisition of ultrasound data, ECG data, and auscultation data, for example, by controlling corresponding electrical circuitry of the ultrasound sensor 46, ECG sensors 48, and auscultation sensors 47 (see FIG. 3A) of the probe 40 in response to selective activation of the selectable icons 71, 72, 73.

In some embodiments, a time scale input element 74 may be displayed and utilized to control a time scale for display of the ECG data 82 or digital auscultation data 83. For example, through user input provided via the slider 31, or buttons 32, the time scale may be adjusted (e.g., by increasing or decreasing the time scale).

In some embodiments, an ECG gain input element 76 and a digital auscultation (DA) gain input element 78 are displayed and utilized to control ECG gain and DA gain, for example, through user input provided by the slider 31 or buttons 32.

In some embodiments, the tablet 20 includes a haptic feedback element, such as a vibrator or the like, which is capable of providing haptic feedback to a user, e.g., in response to a user selection via the user input elements. For example, in some embodiments, the tablet 20 includes a haptic feedback element within the handle 22 which vibrates in response to the user activating (e.g., depressing, scrolling, or the like) one of the user input elements on the handle 22. This lets the user know, via haptic feedback, that input has been received via the user input elements, which may be advantageous when the user is viewing the display 21 and the user's hand on the handle 22 is out of view behind the display 21.

The user input elements on the handle 22 may allow the user to control key imaging or data acquisition functions. The user can use one or more fingers to interact with various control elements (e.g., the user input elements) which are integrated into the tablet 20. Such control elements include but are not limited to push buttons, sliders and touchpads. The user input elements are optimally positioned on the handle 22 for efficient usability and an intuitive user experience.

The tablet 20 includes a plurality of electrical connectors which facilitate electrical and communicative connections to external devices, such as the probe 40. For example, in some embodiments, a probe connector 27 (see FIG. 2E) is included on the handle 22. As shown in FIG. 2E, the probe connector 27 may be included at the back end 61 of the handle 22, such as on a portion of the handle 22 which contacts the base of a user's palm during use. The probe connector 27 may be included on the lower portion 24 of the handle 22. The probe connector 27 may extend into an interior space of the lower portion 24 and may be electrically coupled to circuitry within the tablet 20, such as processing circuitry or the like for processing signals received through the probe connector 27 for display of the signals on the display 21.

The probe connector 27 is configured to electrically couple the probe 40 to the tablet 20. In some embodiments, the probe connector 27 is a USB-C connector or other type of standard or non-standard connector. In some embodiments, the probe connector 27 may be a modified or customized connector in which various input pins, output pins, connections or the like have been modified or customized for connecting circuitry of the probe 40 to circuitry in the tablet 20. The probe connector 27 may be “keyed” such that a corresponding connector on the probe 40 can only mate with the probe connector 27 if properly oriented. To that end, the probe connector 27 may include one or more protrusions 28 which define a correct orientation for connection. The protrusions 28 or keyed features of the probe connector 27 may be formed by the overmold 25, in some embodiments. In some embodiments, the protrusions 28 or keyed features of the probe connector 27 may be formed by the lower portion 24 of the handle 22, for example, the lower portion 24 of the handle 22 may form an opening of the probe connector 27, with the opening having a shape which includes the protrusions 28. In some embodiments, the protrusions 28 may have different shapes or sizes. For example, an upper protrusion 28 may have a shape or size that is different from that of a lower protrusion 28, as shown in FIG. 2E.

The tablet 20 may include a variety of additional features, as depicted in the drawings. For example, the tablet 20 may include one or more cameras 32, a microphone, additional buttons, or the like. In some embodiments, the tablet 20 includes one or more buttons 34 on a side surface or edge of the tablet 20 (see FIG. 2C), which may be utilized to control various parameters of the tablet 20, such as volume, brightness of the display, or the like.

In some embodiments, the overmold 25, which may be a silicone overmold, covers one or more of the buttons 32 on the tablet 20. The overmold 25 may completely seal the buttons 32, and may provide a waterproof seal which protects the buttons 32 (as well any other features which are covered by the overmold 25) from permeation by liquids or other substances, including, for example, water, ultrasound gel, bodily fluids, or the like. In some embodiments, the overmold 25 is hydrophobic. The overmold 25 may be directly formed on the tablet 20, as previously discussed. That is, in various embodiments, the overmold 25 is not a separately formed component that is later attached to the frame 23, but instead, the overmold 25 is formed directly on the frame 23 and forms an integral component of the tablet 20 itself. This advantageously provides features such as ruggedness and liquid-sealing (which protect the buttons and other features of the tablet 20) without having an external case.

FIGS. 3A and 3B are a perspective top view and a side view, respectively, illustrating the clinical data acquisition probe 40 of the clinical data acquisition system 10, in accordance with one or more embodiments.

Referring to FIGS. 3A and 3B, the probe 40 includes an outer housing 44 which may surround internal electronic components and/or circuitry of the probe 40, including, for example, one or more ultrasound transducers, electronics such as driving circuitry, processing circuitry, oscillators, beamforming circuitry, filtering circuitry, and the like. The housing 44 may be formed to surround or at least partially surround externally located portions of the probe 40, such as a sensor face 42, and may be a sealed housing, such that moisture, liquid or other fluids are prevented from entering the housing 44. The housing 44 may be formed of any suitable materials, and in some embodiments, the housing 44 is formed of a plastic material. The housing 44 may be formed of a single piece (e.g., a single material that is molded surrounding the internal components) or may be formed of two or more pieces (e.g., upper and lower halves) which are bonded or otherwise attached to one another.

The probe 40 includes at least one sensor that, in use, acquires physiological data of a patient. In some embodiments, the probe 40 includes an ultrasound sensor 46. In some embodiments, the probe 40 may include one or more electrocardiogram (ECG) sensors and one or more auscultation sensors. For example, U.S. patent application Ser. No. 15/969,632 (now U.S. Pat. No. 10,507,009) and U.S. patent application Ser. No. 16/593,173, assigned to the assignee of the present disclosure and incorporated by reference herein, describe various embodiments of ultrasound probes having one or more of an ultrasound sensor, an auscultation sensor, and an ECG sensor.

As shown in FIG. 3A, the ultrasound sensor 46 is located at or near the sensor face 42. For example, in some embodiments, the ultrasound sensor 46 is located behind the sensor face 42 and may be covered by a material that forms the sensor face 42, such as a room-temperature-vulcanizing (RTV) rubber or any other suitable material. In some embodiments, an ultrasound focusing lens is included at the sensor face 42 and may cover the ultrasound sensor 46. The ultrasound focusing lens may be formed of RTV rubber or any other suitable material.

The ultrasound sensor 46 is configured to transmit an ultrasound signal toward a target structure in a region of interest of a patient, and to receive echo signals returning from the target structure in response to transmission of the ultrasound signal. To that end, the ultrasound sensor 46 may include transducer elements that are capable of transmitting an ultrasound signal and receiving subsequent echo signals. In various embodiments, the transducer elements may be arranged as elements of a phased array. Suitable phased array transducers are known in the art.

The transducer elements of the ultrasound sensor 46 may be arranged as a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 46 may include piezoelectric micromachined ultrasonic transducers (PMUT), which are microelectromechanical systems (MEMS)-based piezoelectric ultrasonic transducers, or the ultrasound sensor 46 may include capacitive micromachined ultrasound transducers (CMUT) in which the energy transduction is provided due to a change in capacitance.

In some embodiments, the probe 40 includes an electrocardiogram (ECG) sensor 48. The ECG sensor 48 may be any sensor that detects electrical activity, e.g., of a patient's heart, as may be known in the relevant field. For example, the ECG sensor 136 may include any number of electrodes 48a, 48b, 48c, which in operation are placed in contact with a patient's skin and are used to detect electrical changes in the patient that are due to the heart muscle's pattern of depolarizing and repolarizing during each heartbeat.

As shown in FIG. 3A, the ECG sensor 48 may include a first electrode 48a that is positioned adjacent to a first side of the ultrasound sensor 46 (e.g., adjacent to the left side of the ultrasound sensor 46, as shown), and a second electrode 48b that is positioned adjacent to a second side of the ultrasound sensor 46 that is opposite to the first side (e.g., adjacent to the right side of the ultrasound sensor 46, as shown). The ECG sensor 48 may further include a third electrode 48c that is positioned adjacent to a third side of the ultrasound sensor 46 (e.g., adjacent to the lower side of the ultrasound sensor 46, as shown). In some embodiments, each of the first, second, and third electrodes 48a, 48b, 48c have different polarities. For example, the first electrode 48a may be a positive (+) electrode, the second electrode 48b may be a negative (−) electrode, and the third electrode 48c may be a ground electrode. The number and positions of the ECG sensor electrodes may vary in different embodiments.

In some embodiments, the probe 40 further includes one or more auscultation sensors 47a, 47b at or adjacent to the sensor face 42, as described, for example, in U.S. patent application Ser. No. 16/593,173, which is assigned to the assignee of the present disclosure and incorporated by reference herein. The one or more auscultation sensors 47a, 47b may be any sensors operable to detect internal body sounds of a patient, including, for example, body sounds associated with the circulatory, respiratory, and gastrointestinal systems. For example, the auscultation sensors 47a, 47b may be microphones. In some embodiments, the auscultation sensors 47a, 47b may be electronic or digital stethoscopes, and may include or otherwise be electrically coupled to amplification and signal processing circuitry for amplifying and processing sensed signals, as may be known in the relevant field.

Each of the ultrasound sensor 46, the ECG sensor(s) 48, and the auscultation sensor(s) 47 may be positioned at or adjacent to the sensor face 42 of the probe 40. In some embodiments, two or more of the ultrasound sensor 46, the ECG sensor(s) 48, and the auscultation sensor(s) 47 may be positioned on a same plane, e.g., coplanar with one another at the sensor face 42 of the probe 40. In use, the sensor face 42 may be placed in contact with a patient's skin, and the probe 40 may obtain ultrasound, ECG, and auscultation signals via the ultrasound sensor 46, the ECG sensor 48, and the auscultation sensor 47, respectively. The probe 40 may obtain the ultrasound, ECG, and auscultation signals sequentially or simultaneously in any combination.

Clinical data acquired by the probe 40, such as ultrasound signals, ECG signals, auscultation signals, or any other clinical data or signals, may be transmitted to the tablet 20 via the cable 12 and a connector 14. The cable 12 may extend from the probe 40 (e.g., from a proximal end of the probe 40) and terminates at the connector 14.

The connector 14 may be sized and configured to electrically couple the probe 40 to probe connector 27 of the tablet 20. For example, the connector 14 may be keyed or otherwise include features which only allow the connector 14 to fit into the probe connector 27 on the tablet 20 if the connector 14 is properly oriented. For example, as shown in FIGS. 3A and 3B, the connector 14 may include one or more grooves 15 sized to accommodate the one or more protrusions 28 of the probe connector 27.

In some embodiments, the connector 14 may include grooves 15 on upper and lower sides of the connector 14, and each of the grooves 15 may be sized to accommodate a corresponding one of the protrusions 28 of the probe connector 27. The grooves 15 of the connector 14 may ensure proper orientation of the connector 14 when inserted into the probe connector 27, as the grooves 15 may allow insertion of the connector 14 into the probe connector 27 in only one orientation. Similarly, the grooves 15 of the connector 14 may prevent the connector 14 from being inserted into any conventional electrical connectors, such as a conventional USB-C connector.

In some embodiments, the signals acquired from the auscultation sensor(s) 47, the ECG sensor(s) 48, and the ultrasound sensor 46 may be simultaneously acquired and synchronized with one another. For example, U.S. patent application Ser. No. 15/969,632, assigned to the assignee of the present disclosure and incorporated by reference herein in its entirety, describes various embodiments of devices, systems, and methods in which auscultation data, ECG data, and ultrasound data, which are derived from signals received by an auscultation sensor, an ECG sensor, and an ultrasound sensor, respectively, are synchronized.

The signal acquisition and synchronization techniques described in U.S. patent application Ser. No. 15/969,632 may be modified and implemented in embodiments of the present disclosure for similarly synchronizing the acquired auscultation, ECG, and ultrasound signals, as well as any acquired ambient noise signals. In some embodiments, the acquired auscultation, ECG, and ultrasound signals may be synchronously displayed on the display 21, for example, as shown in FIG. 2A.

The various embodiments described above can be combined to provide further embodiments. All of the U.S. patent applications and patents referred to in this specification and/or listed in the Application Data Sheet are incorporated herein by reference, in their entirety. Aspects of the embodiments can be modified, if necessary, to employ concepts of the various patents and applications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A mobile clinical viewing device, comprising:
a frame;
a display secured to a first side of the frame; and
a handle secured to a second side of the frame, the handle including:
a plurality of user input elements which, in use, control one or more operations of the mobile clinical viewing device in response to input received from a user.

2. The mobile clinical viewing device of claim 1, further comprising a silicone overmold covering at least a portion of the frame.

3. The mobile clinical viewing device of claim 1 wherein the handle includes a first portion secured to the second side of the frame, and a second portion on the first portion, the first and second portions including different materials.

4. The mobile clinical viewing device of claim 3 wherein the first portion of the handle is formed of aluminum.

5. The mobile clinical viewing device of claim 3 wherein the second portion of the handle is formed of overmolded silicone.

6. The device of claim 1 wherein the plurality of user input elements includes a slider element and at least one button spaced laterally apart from the slider element.

7. The mobile clinical viewing device of claim 1 wherein an outer surface of the handle slopes inwardly toward the display from a back end of the handle to a front end of the handle.

8. The mobile clinical viewing device of claim 1 wherein the display has an inclination angle between 20° and 70° with respect to a surface when the device is placed on the surface with the handle in contact with the surface.

9. A clinical data acquisition system, comprising:
a probe having at least one sensor configured to acquire physiological data of a patient; and
a mobile clinical viewing device communicatively coupleable to the probe, the mobile clinical viewing device including:
a frame;
a display secured to a first side of the frame; and
a handle secured to a second side of the frame, the handle including a plurality of user input elements which, in use, control one or more operations of the mobile clinical viewing device in response to input received from a user.

10. The clinical data acquisition system of claim 9 wherein the handle has an ergonomic shape having one or more curved side regions sized to accommodate a user's hand or fingers.

11. The clinical data acquisition system of claim 9 wherein the mobile clinical viewing device includes a keyed probe connector, and the probe includes a cable having a keyed connector which is sized to fit into the keyed probe connector.

12. The clinical data acquisition system of claim 9 wherein the at least one sensor of the probe includes an ultrasound sensor configured to acquire ultrasound data of the patient.

13. The clinical data acquisition system of claim 12, wherein the at least one sensor of the probe further includes an electrocardiogram (ECG) sensor configured to acquire ECG data of the patient, and an auscultation sensor configured to acquire auscultation data of the patient.

14. The clinical data acquisition system of claim 13 wherein the display of the mobile clinical device is configured to display data representative of at least one of the acquired ultrasound data, the ECG data, or the auscultation data.

15. The clinical data acquisition system of claim 14 wherein the user input elements of the handle are configured to control one or more display settings of the data representative of the at least one of the acquired ultrasound data, the ECG data, or the auscultation data.

16. The clinical data acquisition system of claim 13 wherein the display of the mobile clinical viewing device is configured to display a plurality of selectable icons respectively associated with one of the acquired ultrasound data, the ECG data, or the auscultation data, and the user input elements of the handle are configured to control the one or more operations of the mobile clinical viewing device via selective activation of the plurality of selectable icons.

17. An ultrasound system, comprising:
an ultrasound probe configured to acquire ultrasound data of a patient; and
a mobile clinical viewing device communicatively coupleable to the ultrasound probe, the mobile clinical viewing device including:
a display configured to display ultrasound images based on the acquired ultrasound data of the patient; and
a handle coupled to a back side of the display, the handle including a plurality of user input elements configured to control one or more operations of the mobile clinical viewing device in response to input received from a user.

18. The ultrasound system of claim 17 wherein a gripping surface of the handle of the mobile clinical viewing device includes a silicone overmold.

19. The ultrasound system of claim 17 wherein the plurality of user input elements includes a slider element and at least one button spaced laterally apart from the slider element.

20. The ultrasound system of claim 17 wherein the display has an inclination angle between 20° and 70° with respect to a surface when the device is placed on the surface with the handle in contact with the surface.
